(19) United States
(12) Patent Application Publication
(10) Pub. No.: US 2026/0240449 A1
(43) Pub. Date: Aug. 20, 2026

(54) Detection and Monitoring of Respiratory Conditions with Photoplethysmography (PPG)

(71) Applicant: Imperial College Innovations Limited, London (GB)

(72) Inventors: Harry John Davies, London (GB); Danilo Mandic, London (GB); Nicholas Peters, London (GB)

(21) Appl. No.: 19/099,369

(22) PCT Filed: Jul. 27, 2023
(86) PCT No.: PCT/GB2023/051987

§ 371 Date: Jan. 28, 2025

(30) Foreign Application Priority Data
Jul. 28, 2022 (GB) 2211069.6

(51) Int. Cl. A61B 5/08; A61B 5/00; A61B 5/0295

(52) U.S. Cl. CPC A61B 5/0803; A61B 5/0295; A61B 5/746

(57) ABSTRACT

Disclosed herein is an apparatus for determining a severity of a respiratory condition. The apparatus comprises means for receiving data indicative of an intensity of light detected during a photoplethysmography measurement. The apparatus comprises means for determining an indication of intrathoracic pressure based on the received data. The apparatus further comprises means for determining a severity of a 10 respiratory condition based on the determined indication of intrathoracic pressure. A system comprising the apparatus and a sensor for photoplethysmography measurement is also provided.

### FIELD

This relates to the detection and monitoring of respiratory conditions such as chronic obstructive pulmonary disorder (COPD) using photoplethysmography (PPG). In particular, this relates to a method of determining a severity of a respiratory condition, and an apparatus for performing said method. This also relates to a method of distinguishing between obstructive and restrictive respiratory conditions, and an apparatus for performing said method.

### BACKGROUND

Photoplethysmography (PPG) is a non-invasive, optical measurement method that uses a light source and a photodetector (PPG sensor, which can be a pulse oximeter) at the surface of the skin to measure the amount of emitted light that is reflected back to the photodetector after travelling through various body tissues. PPG intensity data is composed of a pulsatile component (AC signal) and a non-pulsatile component (DC signal). The AC signal is synchronized with the heart and related to arterial pulsation, while the DC signal is related to light absorption due to the tissues, veins, and diastolic arterial blood volume.

As the volume of blood changes during cardiac and respiratory cycles, the intensity of reflected light also changes, allowing these volumetric changes in the blood to be measured; in particular, as the volume of blood increases more light is absorbed by the blood, and less emitted light is reflected back to the photodetector. It has been recognised that time-varying blood volume changes determined using the PPG data can be used to estimate respiration rate. Moreover, research has shown that time-varying respiratory waveforms extracted from PPG data may contain sufficient information to detect chronic obstructive pulmonary disease (COPD).

It is desirable to provide an alternative, robust method for detecting or monitoring respiratory conditions using PPG data.

### SUMMARY

The scope of protection sought for various embodiments of the invention is set out by the independent claims. The embodiments and features, if any, described in this specification that do not fall under the scope of the independent claims are to be interpreted as examples useful for understanding various embodiments of the invention.

According to a first aspect, there is described an apparatus comprising means for: receiving data indicative of an intensity of light detected during a PPG or photoplethysmography measurement; determining an indication of intrathoracic pressure based on the received data; and determining a severity of a respiratory condition based on the determined indication of intrathoracic pressure. The indication of intrathoracic pressure is any parameter or metric which varies in a manner that is linked/related to, or otherwise indicative of, variation in the intrathoracic pressure.

According to a second aspect, there is described an apparatus comprising means for: receiving data indicative of an intensity of light detected during a PPG or photoplethysmography measurement over a respiratory cycle; determining a first indication of intrathoracic pressure during an expiration phase of the respiratory cycle based on the received data; determining a second indication of intrathoracic pressure during an inspiration phase of the respiratory cycle based on the received data; and detecting an obstructive respiratory condition based on a ratio of the first and second indications of intrathoracic pressure.

The indication of intrathoracic pressure can be calculated, estimated, or derived from the PPG data in any suitable manner. The means for determining an indication of intrathoracic pressure based on the received data can comprise means for mathematically manipulating the received data indicative of an intensity of light to determine the indication of intrathoracic pressure. Any form of mathematical manipulation may be used, including e.g. integration, differentiation, and/or component analysis. In some examples, the means for determining an indication of intrathoracic pressure based on the received data comprises: means for determining a signal indicative of a relative change in blood volume from the received data; and means for integrating the signal indicative of a relative change in blood volume with respect to time to determine the indication of intrathoracic pressure.

In some examples, the means for determining a signal indicative of a relative change in blood volume comprises means for conditioning the signal (means for signal conditioning). In some specific examples, means for conditioning the signal comprises: means for normalising the received data; means for zeroing the normalised data; and means for inverting the zeroed data to determine the signal indicative of a relative change in blood volume.

In some examples, the means for determining a severity of a respiratory condition based on the determined indication of intrathoracic pressure comprises: means for comparing the determined indication of intrathoracic pressure to one or more predefined indications having an associated severity; and means for classifying the determined indication of intrathoracic pressure with the severity based on the comparing.

In some examples, the means for determining a severity of a respiratory condition based on the determined indication of intrathoracic pressure comprises: means for calculating the severity, the calculating based on one or more predetermined relationships between indication of intrathoracic pressure and severity.

Some examples include means for causing, based on the determined severity, an output to be provided.

Some examples include means for providing the output to a user of the apparatus.

In some examples, the respiratory condition is an obstructive respiratory condition. In some examples, the obstructive respiratory condition is one of: COPD, asthma, or cystic fibrosis.

The means may comprise: at least one processor; and at least one memory including computer program code, the at least one memory and the computer program code configured, with the at least one processor, to cause the performance of the apparatus.

According to a third aspect, there is provided a system comprising the apparatus of the first and/or second aspect. The system further comprises a sensor for photoplethysmography measurement, the sensor comprising: a light source configured to emit light; and a photodetector configured to detect reflected light, wherein the data indicative of an intensity of light detected during a photoplethysmography measurement is indicative of the detected reflected light. In some examples, the sensor is configured to be worn in an ear of a user.

In some examples, the apparatus and/or sensor further comprises one or more additional sensors configured to output one or more sensing signals. The one or more sensing signals can be indicative of at least one of: electrocardiogram (ECG), blood pressure, heart rate variability (HRV), motion, temperature, or audio. However, any additional physiological and/or environmental or contextual signals can be used. The means for determining an indication of intrathoracic pressure can be further configured to determine the indication of intrathoracic pressure based on the one or more sensing signals. In other words, multi-modal signals can be used to determine an indication of intrathoracic pressure.

According to a fourth aspect, this specification describes an apparatus comprising: at least one processor; and at least one memory including computer program code which, when executed by the at least one processor, causes the apparatus to: receive data indicative of an intensity of light detected during a PPG or photoplethysmography measurement; determine an indication of intrathoracic pressure based on the received data; and determine a severity of a respiratory condition based on the determined indication of intrathoracic pressure.

In a sixth aspect, this specification describes an apparatus comprising: a first receiving module configured to receive data indicative of an intensity of light detected during a PPG or photoplethysmography measurement; a first determining module configured to determine an indication of intrathoracic pressure based on the received data; and a second determining module configured to determine a severity of a respiratory condition based on the determined indication of intrathoracic pressure.

In a seventh aspect, a method performed by one or more processors of an apparatus is provided. The method comprises: receiving data indicative of an intensity of light detected during a photoplethysmography measurement; determining an indication of intrathoracic pressure based on the received data; and determining a severity of a respiratory condition based on the determined indication of intrathoracic pressure.

In some examples, the method further comprises repeatedly receiving, at periodic intervals, subsequent data indicative of an intensity of light detected during a subsequent photoplethysmography measurement. In some examples, the method further comprises, for each subsequent data received: determining an updated indication of intrathoracic pressure based on the subsequent data; and updating the severity of the respiratory condition based on the updated indication of intrathoracic pressure.

In some examples, the method further comprises causing, based on the severity, an output to be provided. In some steps, providing an output comprising outputting an alert to a user.

In an eighth aspect, a method performed by one or more processors of an apparatus is provided. The method comprises detecting an obstructive respiratory condition by: receiving data indicative of an intensity of light detected during a PPG or photoplethysmography measurement over a respiratory cycle; determining a first indication of intrathoracic pressure during an expiration phase of the respiratory cycle based on the received data; determining a second indication of intrathoracic pressure during an inspiration phase of the respiratory cycle based on the received data; and detecting an obstructive respiratory condition based on a ratio of the first and second indications of intrathoracic pressure.

According to a ninth aspect, this specification describes a computer program comprising instructions for causing an apparatus to perform any of the methods described herein. According to a tenth aspect, this specification describes a computer-readable medium (such as a non-transitory computer-readable medium) comprising program instructions stored thereon for performing any of the methods described herein.

In an eleventh aspect, this specification describes an in-ear wearable comprising a PPG sensor. The in-ear wearable may be configured to perform any method as described with reference to the seventh and/or eighth aspects, or the earpiece may be in communication with any apparatus described herein.

Also described herein is an apparatus comprising means for: receiving data indicative of an intensity of light detected during a PPG or photoplethysmography measurement; and determining an indication of lung health based on the received data. One or more respiratory conditions can be detected and/or monitored based on the indication of lung health.

The indication of lung health is any parameter or metric which reflects, or is otherwise indicative of, a condition of a user's lung or lung health. In some examples, the indication of lung health can be an indication of intrathoracic pressure, as described above. In these examples, any process described herein can be used to determine the indication of intrathoracic pressure. In other examples, the indication of lung health can be any other parameter or metric indicative of lung health. For example, aspects of the PPG signal, waveform and/or dynamics that are not mediated by or related to intrathoracic pressure may provide an indication of lung health.

The indication of lung health can be calculated, estimated, or derived from the PPG data in any suitable manner. The indication of lung health may be determined based on one or more mathematical operations performed on the PPG data (e.g. integration, differentiation, component analysis). In other words, the means for determining an indication of lung health based on the received data can comprise means for mathematically manipulating the received data indicative of an intensity of light to determine the indication of lung health.

The indication of lung health may additionally or alternatively be determined based on the application of one or more machine learning models to the PPG data. In some examples, one or more additional physiological or environmental/contextual signals may be used in combination with the PPG data/signals to determine the indication of lung health.

Also described herein is an apparatus comprising means for: receiving physiological signals; and determining an indication of lung health based on the received physiological signals. One or more respiratory conditions can be detected and/or monitored based on the indication of lung health. The physiological signals can be measured or sensed by one or more sensors. The physiological signals can include PPG measurements, ECG, blood pressure, heart rate variability, or body temperature. Any other physiological signals may be used. Environmental or contextual signals (environment temperature, audio signals, motion or acceleration) may also be used.

In some examples, the indication of lung health determined based on the physiological signals comprises an indication of intrathoracic pressure. In these examples, any process described herein can be used to determine the indication of intrathoracic pressure. Optionally, receiving physiological signals can comprise receiving data indicative of an intensity of light detected during a PPG or photoplethysmography measurement over a respiratory cycle.

### LIST OF FIGURES

FIG. 1A and FIG. 1B schematically illustrate the principles underlying PPG measurements as a proxy for intrathoracic pressure during expiration for healthy and obstructed lungs, respectively, and FIG. 1C schematically illustrates the principles underlying PPG measurements as a proxy for intrathoracic pressure during a respiratory cycle;

FIGS. 2A and 2B schematically illustrate an example in-ear apparatus for PPG measurements;

FIGS. 3A and 3B schematically illustrate an example apparatus and system, respectively, for performing the methods described herein;

FIGS. 4A and 4B illustrate the change in normalised blood volume during respiration for restrictive and obstructive respiratory conditions, respectively;

FIG. 5A illustrates the summed change in estimated blood volume during expiration and inspiration for restrictive (IPF) and obstructive (COPD) respiratory conditions, and

FIG. 5B shows a ratio between the expiration and inspiration phases for IPF and COPD;

FIG. 6 shows exemplary raw PPG data;

FIG. 7 illustrates a relationship between the normalised summed blood volume over time estimated from PPG data and FEV1/FVC ratio obtained from spirometry measurements;

FIG. 8 illustrates a relationship between different FEV1/FVC ratios and the summed blood volume over time estimated from PPG data;

FIG. 9 shows example waveforms for the estimated blood volume over time and an associated FEV1/FVC value for each waveform; and

FIG. 10 is a flowchart showing example method steps for determining a severity of a respiratory condition.

### DETAILED DESCRIPTION

Respiration comprises two phases: inspiration (inhaling) and expiration (exhaling). When we inspire, the chest cavity expands causing a decrease in intrathoracic pressure (pressure within the chest cavity), which pulls air into the lungs. This decrease in intrathoracic pressure reduces pressure on the heart and veins: both right atrial pressure (RAP) and central venous pressure (CVP) therefore decrease. This causes an increase in venous return, reducing the volume of blood in the venous capillary beds. This reduction in blood volume can be detected with a PPG sensor (also called herein a detector or probe) through a change or modulation in the DC component of the PPG signal. The PPG sensor emits light through the skin into the blood and measures how much light is reflected back to the sensor. Blood absorbs light, and thus if less blood is present then less light is absorbed and more light returns (is reflected back) to the sensor. PPG measurements/data are therefore a proxy for changes in blood volume (relative changes, not absolute blood volume).

Right ventricular stroke volume also increases with the increased venous flow to the heart, sending more blood to the lungs for the uptake of oxygen. In turn, left ventricular stroke volume is decreased, leading to a decreased pulse amplitude (observed through changes in the AC component of the PPG signal). This is accompanied by an increase in heart rate, which causes a decrease in the interval between pulses in the PPG signal (again, observed through changes in the AC component of the PPG signal).

Opposite effects are observed during expiration: the chest cavity contracts, increasing the intrathoracic pressure and causing a reduction in venous return, which leads to an increase in volume of blood in the venous capillary beds.

Three major respiratory modes are therefore present in PPG data during respiration:
1) Respiratory induced intensity variations (RIIVs) which are generated by changes in venous pressure. These RIIVs modulate the DC component of photoplethysmography, and are therefore accessible directly from the raw-PPG signal/data;
2) Pulse amplitude variations due to changes in left ventricular stroke volume, which can be obtained from the envelope of the AC filtered PPG signal; and
3) Pulse interval variations generated through respiratory sinus arrhythmia, which can be obtained by measuring the interval between consecutive pulses in the AC component of the PPG signal.

Respiration rates have previously been estimated from PPG sensors located on a person's forearm, wrist, earlobe, forehead, neck, and chest. The PPG data from such sensors yields high respiration rate accuracy in all three PPG respiratory modes, in both healthy subjects and subjects with breathing disorders (or respiratory conditions), such as chronic obstructive pulmonary disease (COPD) and asthma.

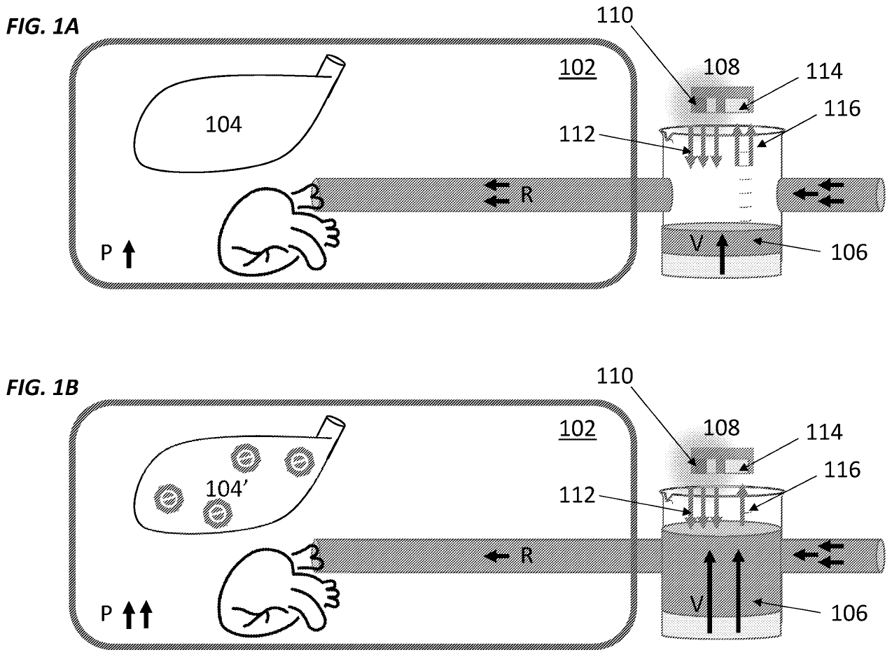

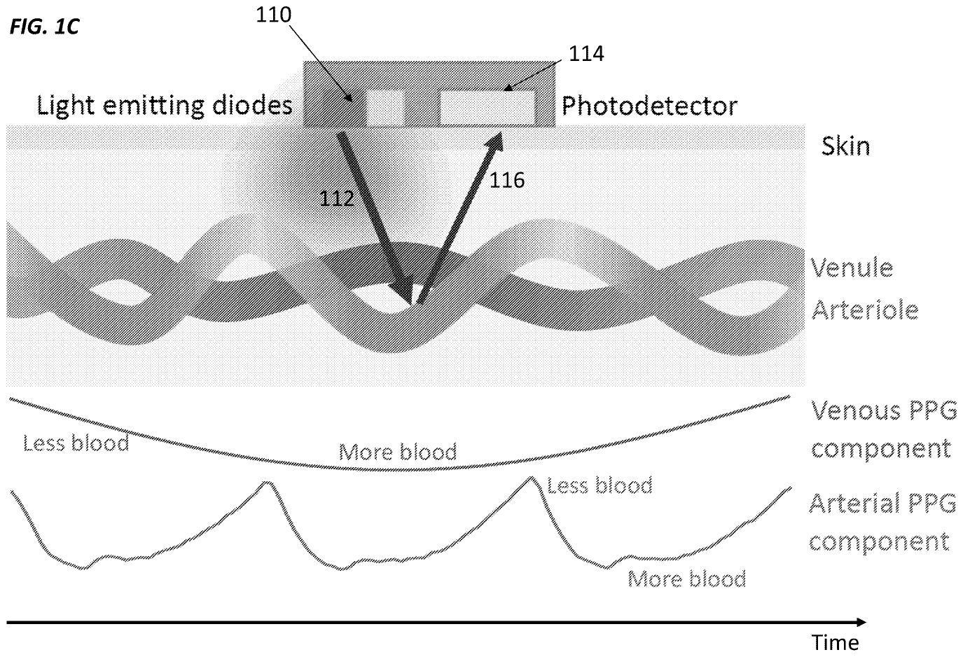

Previous analysis of PPG data has focused on extracting time-varying respiratory waveforms from the data, and detecting or classifying respiratory conditions based on differences in these respiratory waveforms as compared to waveforms from healthy persons (e.g. persons without a respiratory condition). However, it is now recognised that the relationship between blood volume and intrathoracic pressure can be used to directly detect and monitor respiratory conditions from the PPG data. In particular, by using the blood volume as a proxy for intrathoracic pressure, the amount, or size, of blood volume change derived from the PPG data can be used to detect and monitor respiratory conditions. This blood volume-based approach is outlined below with reference to FIGS. 1A and 1B. As discussed above, references herein to blood volume and blood volume change are relative estimations, not absolute values.

FIG. 1A provides a schematic illustration of the thoracic cavity 102 during a normal expiration (exhale). As the diaphragm relaxes, the chest or thoracic cavity 102 reduces in size, causing an increase in intrathoracic pressure P which drives the air out of the lungs 104. As discussed above, this increased pressure leads to a reduction in venous return R within the thoracic cavity, causing the peripheral venous capillary beds to fill slightly with blood (i.e. there is an increase in the volume V of blood 106 within the venous beds). This increase in blood 106 volume in the venous beds is illustrated schematically by the beaker to the right of the thoracic cavity 102 in FIG. 1A.

A detector/sensor 108 is positioned to detect the change in blood volume. The detector comprises a light source 110 configured to emit light 112 and a photodetector 114 configured to detect light 116. In this instance, a portion of emitted light 112 is absorbed by the blood 106, and a portion of light 112 is reflected back as light 116 and detected by the photodetector 114. The amount of reflected light 116 indicates the volume V of blood 106 gathered in the venous beds. This volume V is a function of the venous return R, which is itself a function of the intrathoracic pressure P.

During the inspiration (inhaling) portion of the respiration, intrathoracic pressure decreases and the venous return increases, draining the peripheral venous beds of blood 106. This reduction in the volume of blood 106 increases the amount of reflected light 116 during the inspiration phase; conversely, the amount of reflected light decreases during the expiration phase as the venous beds fill with blood. The amount (or intensity) of reflected light 116 detected by the sensor 108 is therefore indicative of a volume of blood. Since the blood volume V is related to the intrathoracic pressure P, the blood volume derived from the intensity of the PPG waveform can be used as a proxy for the intrathoracic pressure P, and thus how hard a patient is breathing.

In contrast to the normal/healthy expiration of FIG. 1A, FIG. 1B provides a schematic illustration of expiration in a patient or person with obstructed lungs or obstructed airflow (such as occurs with chronic obstructive pulmonary disease). Chronic obstructive pulmonary disease (COPD) is a debilitating illness caused by an increased inflammatory response in the lungs which leads to obstructed airflow, particularly during expiration. However, COPD is used herein as an example of an obstructive respiratory condition only. Similar mechanisms occur for other obstructive respiratory conditions, such as: chronic bronchitis, asthma, bronchiectasis, bronchiolitis, or cystic fibrosis.

Due to the obstructed lungs 104′, a larger increase in pressure P is required to drive the air out of the lungs 104′ during expiration (the patient has to breathe out harder, generating more pressure). This leads to a larger decrease in venous return R than with normal/healthy expiration, and correspondingly a larger increase in the volume V of blood 106 within the peripheral venous capillary beds. This increase in blood volume causes more of the emitted light 112 to be absorbed, leading to a reduction in reflected light 116 (and thus a decrease/reduction in the intensity of light being detected at the photodetector 114 of the sensor 108 as compared to the example of FIG. 1A).

This principle is illustrated further in FIG. 1C, which shows the variation in the magnitude of the measured PPG signal (variation in intensity of reflected light) due to both venule and arteriole blood volume changes. The venule blood volume provides the DC component of the PPG signal, and the arteriole blood volume provides an AC component to the PPG signal. It can be seen that “more blood” (i.e. more blood in the venous beds) corresponds to a lower PPG value (e.g. less light from light source 110 being detected at the photodetector 114), and vice versa.

The blood volume information contained in the PPG data can thus be used as a proxy for intrathoracic pressure, and by extension, as a proxy for respiratory condition. This facilitates provision of a wearable, non-invasive apparatus for diagnosis of COPD and other obstructive respiratory conditions. Moreover, the severity of said conditions can be estimated and monitored based on the PPG data. For example, the greater the obstruction in the lungs, the greater the intrathoracic pressure that is needed to exhale, resulting in a larger decrease in venous return and thus a larger increase in venous blood volume accumulating in the peripheral venous beds (as in the beaker analogy of FIG. 1); in response, more light emitted from the PPG sensor is absorbed by the blood, and less light is reflected back to the sensor and recorded/detected. There is an inversely proportional relationship between the DC component of the light measured by the PPG sensor device and the blood volume.

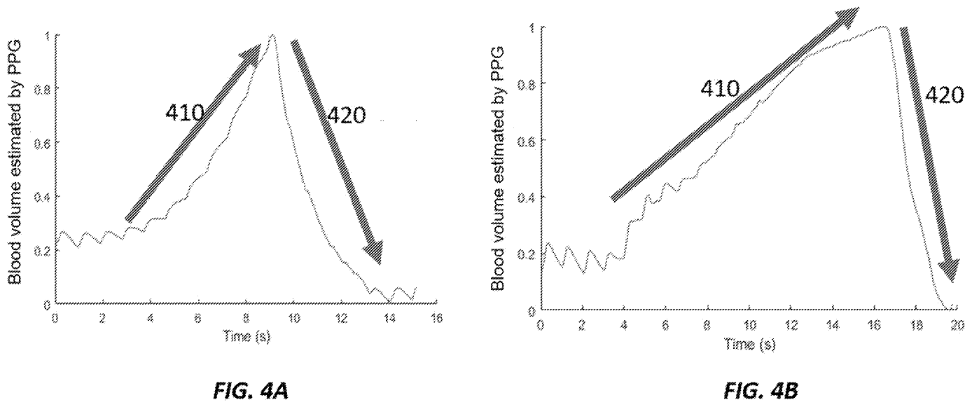

In other words, when the level or severity of obstruction increases, there is a corresponding increase in the summed pressure across time required for each breathe—this increase in the summed pressure over time is reflected in an increase in the summed (or integrated) volume of blood, which can be estimated using wearable PPG sensors, as discussed below with reference to FIG. 4. In particular, there is a decrease in PPG intensity over time that is proportional to the level of the obstruction (and thus the severity of the disease).

The approach described herein further allows such inferences about the severity of respiratory conditions to be made using data from consumer wearables gathered as a user exercises or during their daily life. As such, it is possible to measure changes in the PPG data over different time periods (hourly, daily, weekly, etc.), facilitating the real-time monitoring of a person's condition and allowing changes in severity and life threatening exacerbations to be detected or recognised.

Any suitable sensor device may be used for PPG measurement, and the device may be positioned at any suitable location on/around a user's body to direct light onto a user's skin and detect reflected light, as illustrated in FIG. 1C. The measuring/sensor device includes the sensor 108 discussed above. In some specific implementations, at least part of the sensor device can be provided in the form of an in-ear wearable (apparatus configured to be inserted within an ear canal of a user); such an in-ear apparatus may also be referred to as a “hearable”. However, the device is not limited thereto and any suitable measuring/sensor device at any suitable PPG recording site may be used for taking/recording the PPG measurements.

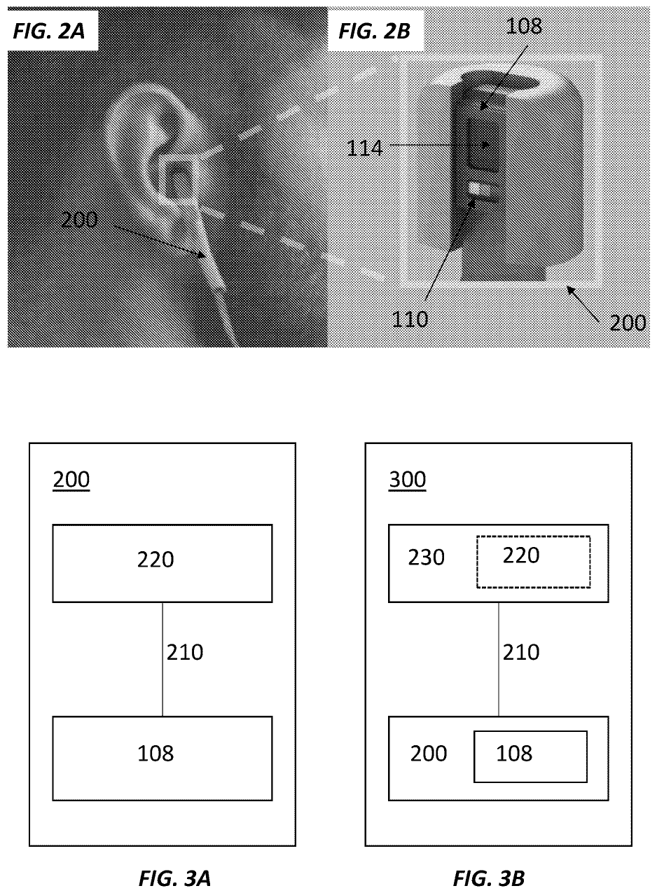

With the growing popularity of in-ear wearable apparatus (so called hearables), the in-ear location is rapidly emerging as a favourable site for wearable photoplethysmography (as it has for ECG measurements previously). PPG data measured from the ear canal is more sensitive to intensity variations than the finger PPG, and also is less obtrusive for a user during their daily life. It is therefore a suitable sensor location for measuring PPG data for the purposes of detecting and monitoring respiratory conditions using the approach described herein. The use of such an in-ear wearable for detecting and monitoring respiratory conditions (or breathing disorders) is described with reference to the specific example of FIG. 2.

With reference to FIG. 2B, an example sensor device 108 is shown comprising light source 110 and photodetector 114. The sensor 108 is provided as part of an apparatus 200. In this example, apparatus 200 is an in-ear wearable 200, or hearable 200, as shown in FIG. 2A. However, it will be understood that this is a specific, example, arrangement and the disclosure is not limited to this particular implementation. Sensor 108 may be provided in any other suitable arrangement or apparatus, and need not be inserted into the ear. The sensor 108 may be positioned at any suitable part of the body for PPG measurement. For example, a finger probe comprising sensor 108 could be used.

In some implementations of FIG. 2, such as those described with reference to FIG. 3A, apparatus 200 further comprises one or more processors 220 for receiving a signal indicative of an intensity of light detected by the photodetector 114 of the apparatus 200, or for receiving a signal otherwise based on said detected intensity. The signal can be received directly from the sensor 108 via a connection 210, or may be received indirectly from the sensor 108 via connection 210 and by way of one or more intermediary processors, controllers, or other components (not shown here). For example, the signal may be received from sensor 108 by way of a microprocessor, or a microcontroller or microcontroller unit (MCU). In other words, sensor 108 and processor(s) 220 are provided within a single apparatus 200—the sensor device and processors are integral with the apparatus (optionally, integral with the in-ear wearable when apparatus 200 is provided as such).

The one or more processors 220 are configured to perform the methods described herein. For example, the one or more processors are configured to determine an indication of intrathoracic pressure based on the signal indicative of an intensity of light (PPG data). Apparatus 200 comprising processors 220 is one specific example of an apparatus comprising means configured to perform the method described herein. In other examples, any other suitable means may be provided to perform the methods described herein. Processors 220 may also be in communication with one or more other apparatus/devices remote from the apparatus 200 (not shown here).

In some examples, the apparatus 200 further comprises one or more sensors, each configured to output sensing signals. The sensing signals can be physiological and/or environmental/contextual signals. The sensing signals can be indicative of ECG, blood pressure, heart rate variability (HRV), motion (i.e. as detected by an accelerator or IMU), temperature, or audio (i.e. sound signals recorded by a microphone). All these sensors can be included as part of apparatus 200 (optionally, included in or integral with the in-ear wearable when apparatus 200 is provided as such). Alternatively, these sensors can be external sensors (external to the apparatus). For example, microphones on smart devices/tablets or other computing devices may be used, as may stationary temperature sensors, etc. In some specific implementations, the sensors may be provided within any other wearable associated with a user of the apparatus 200. In other words, multi-modal signals, which can be readily detectable by sensors of wearables or hearables, can be used in combination with the PPG data to determine the indication of intrathoracic pressure.

In other examples, the sensing signals output by the one or more sensors may be used in place of, or instead of, the PPG data to determine an indication of lung health (such as an indication of intrathoracic pressure or other metric/parameter). In some examples, physiological signals may be received from the one or more sensors, and an indication of lung health determined based on the received physiological signals. One or more respiratory conditions can be detected and/or monitored based on the indication of lung health. Environmental signals may also be used instead of, or in combination with, the physiological signals.

In other implementations described with reference to FIG. 3B, a system 300 comprises an apparatus 200 having sensor 108 and a computing device 230, where the computing device is provided remote from the apparatus 200. The computing device 230 may be a mobile computing device (e.g. a mobile device, a smartphone, a tablet, a laptop, etc.), or any other computing device/apparatus remote from the apparatus 200. The computing device 230 is configured to receive a signal indicative of an intensity of light detected by the photodetector 114 of the apparatus 200, or a signal otherwise based on said detected intensity. The signal can be received directly from the apparatus 200 via connection 210, or may be received indirectly via connection 210 and by way of one or more intermediary processors, controllers, or other components (not shown here).

In some examples, computing device comprises one or more processors 220 configured to perform the methods described herein. For example, the computing device is configured to determine an indication of intrathoracic pressure based on the signal indicative of an intensity of light (PPG data). The computing device 230 comprising processors 220 is one specific example of an apparatus comprising means configured to perform the method described herein. In other examples, any other suitable means may be provided to perform the methods described herein. In some examples, the system 300 further comprises one or more sensors, each configured to output sensing signals. As discussed above, these sensors can be included as part of apparatus 200 and/or can be provided within any other wearable associated with a user of the system 300. The sensing signals output by the sensors can be used in combination with the PPG data to provide a multi-modal approach to determining the indication of intrathoracic pressure.

Connection 210 can be a wired connection or a wireless connection, as appropriate. In other words, the signal can be received using any suitable communication protocol, and over any suitable connection or network arrangement. For example, wireless embodiments may be deployed in 2G/3G/4G/5G networks and further generations of 3GPP, but also in non-3GPP radio networks such as Wi-Fi. Embodiments may also use Bluetooth, for example. Names of network elements, protocols, and methods are based on current standards. In other versions or other technologies, the names of these network elements and/or protocols and/or methods may be different, as long as they provide a corresponding functionality.

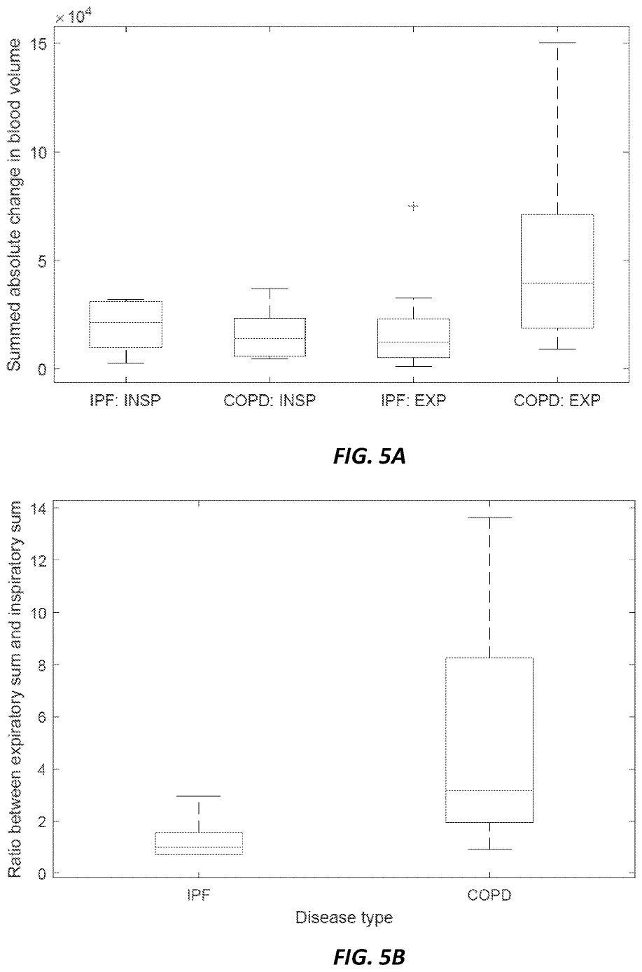

With reference to FIGS. 4 and 5, the use of an indication of intrathoracic pressure to diagnose the presence of an obstructive breathing disorder, infer the severity of an obstructive breathing disorder and monitor exacerbations in individuals is discussed.

In the implementations illustrated in FIGS. 4 and 5, the indication of intrathoracic pressure is defined as the summed, or integrated blood volume over time. The calculated sum/integral combines an amplitude and duration of the measured PPG signal, and thus of the blood volume change. This indication of intrathoracic pressure (as estimated from the relative blood volume changes reflected in the measured PPG data) is a proxy for the intrathoracic pressure itself. However, in other implementations, the amplitude or duration of the estimated blood volume change can be used individually (or independently) to determine an indication of intrathoracic pressure. Moreover, it will be understood that the PPG signal can be mathematically manipulated in other ways to provide an indication of intrathoracic pressure (e.g. by performing differentiation or component analysis).

It will also be understood that, in other examples, the PPG data may be used to directly provide an indication of intrathoracic pressure without first estimating the blood volume changes. For example, the intensity of light measured at the PPG sensor device may be directly mapped or linked to a severity of a respiratory condition. In some implementations, the amplitude and/or duration of the PPG signal can be used. Alternatively, any other suitable metrics or values derived from the PPG signal can be used. In other words, the PPG signal can be used to determine an indication of lung health using any suitable processing techniques. The indication of lung health may or may not be related to the intrathoracic pressure.

FIG. 4 shows relative blood volume changes estimated from measured PPG light intensity waveforms during respiration. In particular, FIG. 4 illustrates a schematic change in blood volume estimated from measured PPG intensity (i.e. blood volume estimated from a signal indicative of the change in the intensity of detected light 116 at photodetector 114) during respiration.

For comparative purposes, FIG. 4 shows a normalised estimated blood volume, with ‘1’ representing the maximum blood volume and ‘o’ representing the minimum blood volume. The change in blood volume estimated from the PPG data is illustrated as an individual exhales as hard as possible, followed by an inhale: changes in blood volume are shown for a typical patient with a restrictive breathing condition (here idiopathic pulmonary fibrosis, IPF) in FIG. 4A, and for a typical patient with an obstructive breathing condition (here COPD) in FIG. 4B.

As illustrated by arrows 410, during expiration/exhalation the venous beds fill up with blood 106 due to increased thoracic pressure. Less light is therefore detected at the photodetector 114 at the end the exhalation, and there is a decrease in raw PPG intensity (corresponding to an increase in blood volume). Similarly, as illustrated by arrows 420, during inhalation/inspiration the venous beds empty of blood 106 as the thoracic pressure decreases. More light is therefore detected at the photodetector 114 at the end of the inhalation, and the raw PPG intensity increases (corresponding to a decrease in blood volume). The PPG intensity—and subsequent estimated blood volume change-is therefore a proxy for thoracic pressure; a lower PPG intensity (representative of a lower amount/intensity of reflected light 116) correlates to a higher volume of blood and thus to a higher thoracic pressure.

If someone suffers from airway obstruction (as in the case of COPD or asthma, for example), during exhalation the thoracic pressure is increased for a longer period of time (in order for the individual to fully exhale) as compared to an individual with a restrictive respiratory condition, or an individual without a respiratory condition. In other words, a respiratory exertion (reflecting how hard an individual has to breathe, and defined as the summed or integrated pressure across time required for each breath) is greater during exhalation for individuals with obstructive respiratory conditions/disorders. Correspondingly, the increase in blood volume in the venous beds is greater during exhalation for individuals with obstructive respiratory conditions/disorders than those with restrictive ones.

Therefore, this change in respiratory exertion during exhalation can be seen in the corresponding increase in blood volume (estimated from the PPG intensity) over time in FIG. 4B as compared to FIG. 4A. The change in blood volume is proportional to the level of the obstruction (and thus the respiratory exertion required to exhale), and is thus representative of the severity of the disease. For distinguishing between restrictive (FIG. 4A) and obstructive disease (FIG. 4B), the relationship between the increase in blood (decrease in intensity, expiration, slope 410), and the subsequent decrease in blood (return to normal intensity, inspiration, slope 420) can be considered. For restriction based respiratory conditions there can be an increased change in blood volume during a breath as compared to a healthy individual, but the balance between inspiration 420 and expiration 410 is very similar, as shown by the estimated blood volume in FIG. 4A.

In contrast, for COPD and other obstructive based respiratory conditions, a user has to breathe out harder so a greater duration of the respiration cycle or breath is expiration phase 410 (i.e. the portion of the respiratory cycle in which the blood volume increases). The respiratory cycle is therefore skewed (not balanced between expiration and inspiration), as shown in FIG. 4B. By considering this ‘balance’ (e.g. by mathematically manipulating, such as by summing or integrating the blood volume during the inspiration and expiration phases, respectively, and then comparing the values), an obstructive condition can be distinguished from a restrictive one.

This approach for distinguishing between obstructive (here COPD) and restrictive (here IPF) lung diseases can be seen in FIG. 5A, which clearly shows an increase in the summed or integrated blood volume (e.g. an increase in the indication of intrathoracic pressure) on the exhale for individuals with COPD as compared to IPF (using an arbitrary unit). Patients were asked to breathe in and out as hard as possible, and the indication of intrathoracic pressure was calculated using the PPG data, where INSP indicates the inspiration/inhale phase of the respiration (e.g. corresponding to arrows 420 of FIG. 4) and EXP indicates the expiration/exhale phase (e.g. corresponding to arrows 410 of FIG. 4). A comparison of the ratio between expiratory sum and inspiratory sum is shown in FIG. 5B, which illustrates the difference in ‘balance’ between the respiratory phases for restrictive (IPF) and obstructive (COPD) conditions.

In other words, an obstructive respiratory condition can be detected or determined from the indication of intrathoracic pressure. Detecting an obstructive condition first comprises receiving data indicative of an intensity of light detected during a photoplethysmography measurement over a respiratory cycle. Then, a first indication of intrathoracic pressure is determined during an expiration phase of the respiratory cycle based on the received data, and a second indication of intrathoracic pressure is determined during an inspiration phase of the respiratory cycle based on the received data. An obstructive respiratory condition is determined or detected based on a ratio (or ‘balance’) of the first and second indications of intrathoracic pressure.

As discussed above, by recognising that PPG data can be a proxy for intrathoracic pressure, an indication of intrathoracic pressure can be calculated using the measured PPG data. This indication or metric can be obtained in any suitable way.

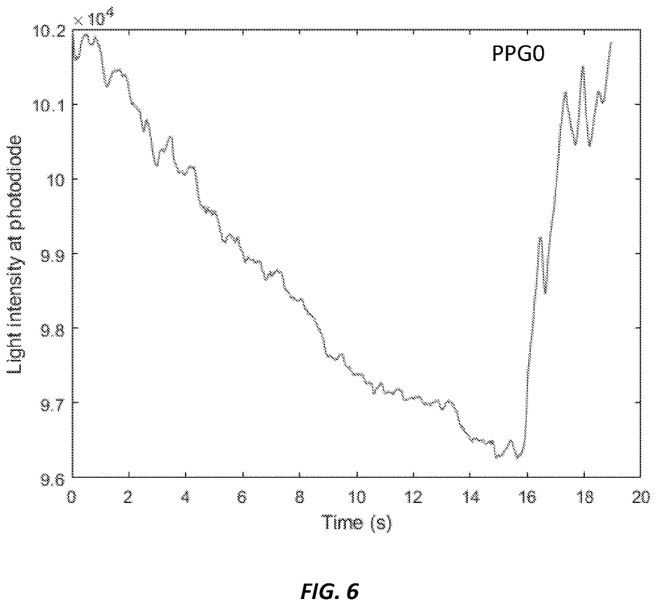

Signal conditioning can be performed to obtain the summed/integrated blood volume. To obtain the summed (or integrated) blood volume (an example of an indication of intrathoracic pressure) shown in the specific example of FIG. 5A, the raw PPG signal (PPG0) is normalised (n-PPG0) with respect to the maximum measured value and then zeroed to just before the start of a breath: PPG1=n−PPG0'PPG0 (t=1). An example of the raw PPG0 signal is illustrated in FIG. 6 (which uses an arbitrary unit). This PPG1 signal is then inverted, so that decreases in PPG intensity are now positive (corresponding to the increase in blood volume, and thus the increase in thoracic pressure): PPG2=(−1)*(PPG1). PPG2 thus reflects the relative change in blood volume. The data shown in FIG. 4 is illustrative of the PPG2 signal. The sum of the PPG2 signal is then taken from the start to the end of the breath. In other words, the PPG2 signal is integrated over the time between the start and end of a single breath (e.g. integrated over the duration of the respiratory cycle 410, 420, as illustrated in FIG. 4). In some examples (not shown here), the raw PPG signal PPG0 is obtained by first filtering or smoothing the raw data from the PPG sensor to remove the pulsatile component of the PPG signal (e.g. signal components due to arteriole volume changes).

COPD has previously been diagnosed with spirometry, by measuring the ratio of volume during forced expiration in one second (FEV1) against forced vital capacity (FVC). Some severity definitions may also use the FEV1 value only. COPD can be diagnosed through an FEV1/FVC of less than 0.7, where a lower FEV1/FVC ratio is indicative of a more severe disease. Decline in the FEV1/FVC over time is indicative of increased obstruction of the lungs, and can therefore indicate a more severe disease. COPD severity can therefore be monitored over time. By simultaneously taking PPG measurements (from sensor 108) and spirometry measurements (from a patient blowing into a tube), the FEV1/FVC can be compared to the change in blood volume over time. It has been found that the change in blood volume can indicate an FEV1/FVC ratio, as discussed below in more detail.

In the following examples, PPG measurements were taken during spirometry measurements. Spirometry measurements consists of a full exhale as hard and as fast as the patient can, continuing until lungs are empty; this is why the resulting waveforms can regularly span over 10 seconds in the following discussion and data. However, regular (or tidal) breathing cycles are generally over a shorter time span. The same principles and phenomena described herein also occur during tidal breathing, and PPG measurements taken independently of spirometry measurements can instead be used to determine the indication of intrathoracic pressure. In other words, the principles described herein can be performed during regular breathing of a user or patient. In this way, real time detecting and monitoring of respirator conditions can be facilitated.

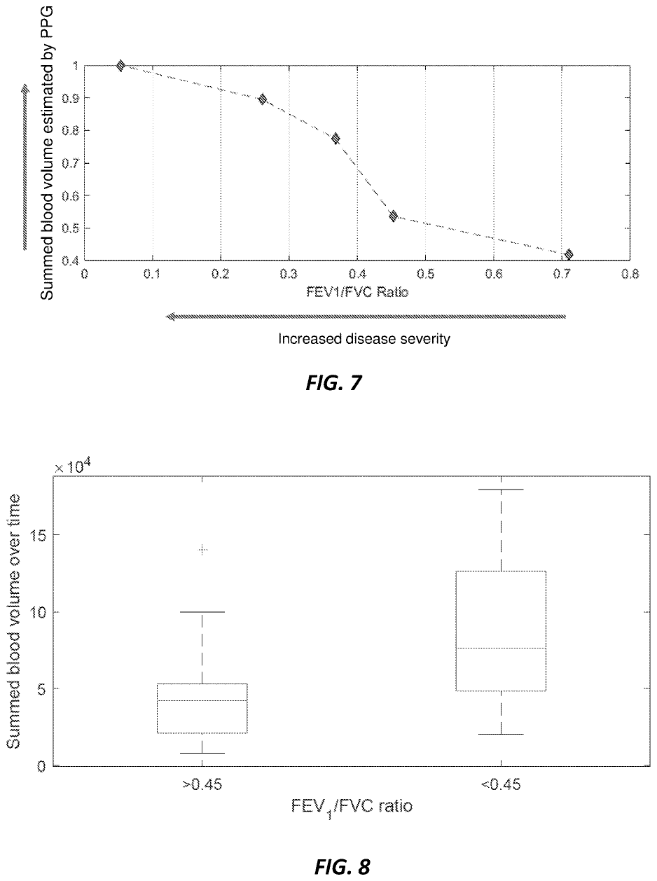

It has been recognised that, for any given user or patient, there is a relationship between the summed (or integrated) change in blood volume and the FEV1/FVC ratios which are typically used to diagnose/characterise COPD. An example of such an individual relationship is shown in FIG. 7, where the summed change in blood volume is shown with arbitrary units for comparative purposes. Here, obstruction was increased artificially, by having the subject breathe into a tube-based apparatus where the expiratory tube diameter was progressively decreased to mimic increased obstruction in the lungs. In other words, the blood volume change as determined from PPG data can be used instead of spirometry measurements to indicate the severity of COPD. This relationship also applies to the prediction of FEV1 alone; varying the tube diameter (or obstruction) does not impact on lung capacity, so the FVC value is constant in FIG. 7 for all of the ratios shown.

On an individual basis, it can be seen from FIG. 7 that when an obstruction gets worse (FEV1/FVC ratio decreases, or FEV1 decreases), the estimated blood volume change increases. The greater the summed blood volume, the greater the severity of the disease. An increase in the sum of the blood volume thus indicates a worsening in condition/exacerbation of the disease. This relationship could be calibrated to an individual to give specific FEV1/FVC ratios for an estimated integrated blood volume change, allowing monitoring of a respiratory condition over time. However, the change in summed blood volume can also generally indicate that there is a change in obstruction, and provides a different way of measuring, classifying or detecting this change.

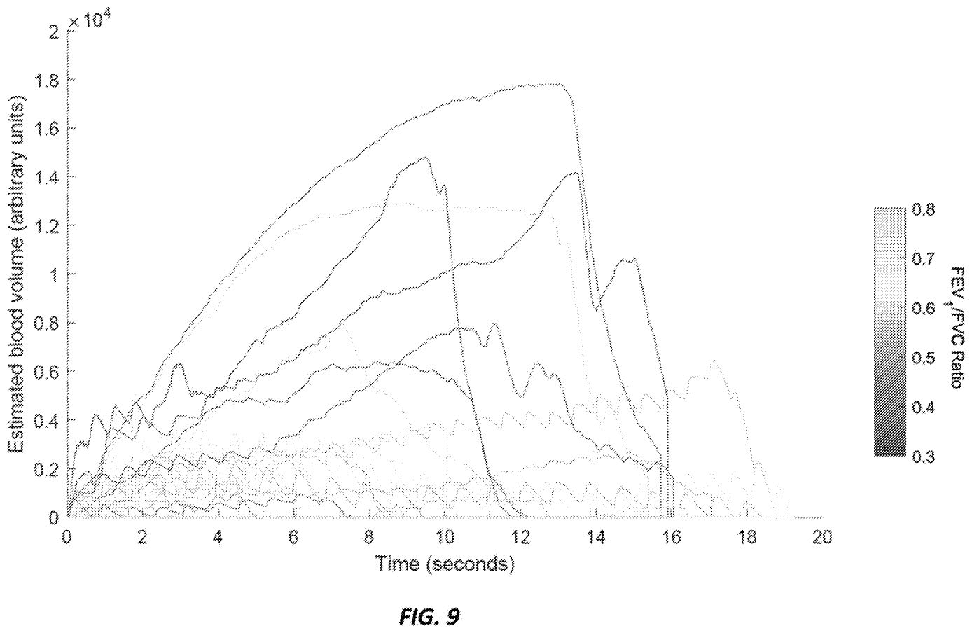

This approach is discussed further with respect to FIGS. 8 and 9. FIG. 9 illustrates relative increases in the venous blood (PPG2 signal) across a variety of different people. The waveforms in FIG. 9 are obtained from raw PPG data (such as the exemplary data shown in FIG. 6, here the raw rata is labelled PPG0) in the manner described above. The waveforms can also optionally be scaled, or normalised, relative to their starting amplitude (blood volume before the breathing cycle begins); alternatively, any other suitable normalisation value may be used. In other words, since the relative blood volume is being considered and compared, not the absolute value, the waveforms can be manipulated in any suitable manner.

The integral of these FIG. 9 waveforms (or the integrated blood volume change) is shown in FIG. 8 (which uses an arbitrary unit). Spirometry measurements were taken at the same time as the PPG measurements, allowing the waveforms in FIG. 9 to be classified with a corresponding FEV1/FVC ratio. Considering the integral of these waveforms in FIG. 8, it can be seen that the summed increase in blood volume is greater for FEV1/FVC ratios>0.45 than for FEV1/FVC ratios<0.45. The differences in the determined integrals is therefore enough to distinguish low FEV1/FVC ratios from high FEV1/FVC ratios (and so distinguish severe obstruction from mild obstruction). The summed increase in blood volume can thus be used in place of the FEV1/FVC ratio to classify the severity of a disease. By using PPG data to determine the summed increase in blood volume, real time monitoring may be performed.

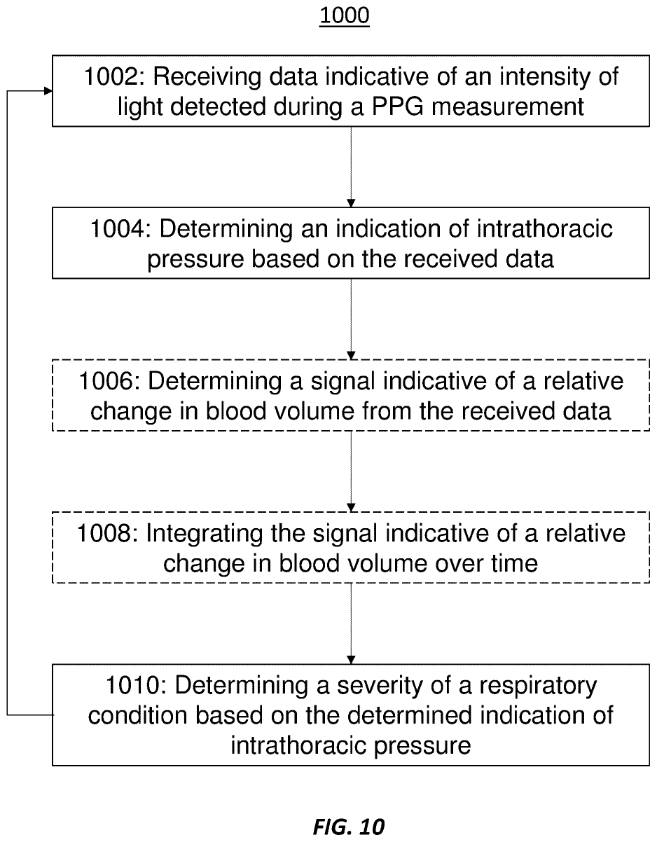

With reference to FIG. 10, an example method 1000 performed by apparatus 200 or computing device 230 of system 300 is described. Example method 1000 can be performed by any apparatus comprising means for performing the method.

S1002 comprises receiving data indicative of an intensity of light detected during a photoplethysmography or PPG measurement. The data indicative of the intensity of light (i.e. the PPG data) can be the raw PPG signal (PPG0) or other data indicative of this raw signal (for example, the signal may be e.g. compressed, modulated, filtered, or otherwise processed before being received at step 902). The measurement can be performed on a user in real time, or the data may be from previously performed measurements. The measurement can be performed with a sensor remote from an apparatus at which the operations described herein are performed. For example, the data can be transmitted from sensor 108 to apparatus 200, where apparatus 200 performs the operations of method 1000.

S1004 comprises determining an indication of intrathoracic pressure based on the received data. The indication of intrathoracic pressure is a parameter indicative of an intrathoracic pressure of a user during a respiration cycle (or during more than one cycle, as appropriate). In other words, the indication of intrathoracic pressure reflects how hard a user has to breath. The indication of intrathoracic pressure can be calculated, estimated, or derived from the PPG data in any suitable manner.

In some optional implementations, the indication of intrathoracic pressure is defined as an integral (or summation) of the relative blood volume change. In such implementations, S1004 can comprise step S1006 and S1008 (shown in the dashed boxes of FIG. 10 as optional features).

Step 1004 comprises determining a signal indicative of a change in blood volume from the received data. Due to the relationship between reflected light intensity and blood volume changes, the data received at step 1002 can be processed to provide a signal indicative of a relative change (relative increase and/or decrease) in blood volume (i.e. not an absolute volume). In some optional implementations, determining a signal indicative of a relative change in blood volume may comprise normalising the received data PPG0. The received data PPG0 can be normalised (n−PPG0) using a maximum value, or any other suitable normalisation parameter. Determining a signal indicative of a relative change in blood volume may comprise zeroing the normalised data n-PPG0 to form zeroed data PPG1, where PPG1=n−PPG0−PPG0(t=1). The zeroing is described with respect to a start of a breath, t=1, but any other suitable zero point can be taken. Determining a signal indicative of a relative change in blood volume may comprise inverting the zeroed received data to form signal PPG2, where signal PPG2 is a signal indicative of a relative change in blood volume and PPG2=(−1)*(PPG1). In other words, the PPG1 signal is inverted, so that decreases in PPG light intensity are now positive (corresponding to increases in blood volume). The PPG light intensity data PPG0 has thus been transformed to data indicative of a change in blood volume PPG2 based on the relationship between absorbed light and blood volume.

S1006 comprises integrating the signal indicative of a relative change in blood volume with respect to time (i.e. over a time period) to determine the indication of intrathoracic pressure. The PPG2 signal of step S1004 corresponds to a change in relative blood volume present in the venous beds (which is itself influenced by intrathoracic pressure). The indication of intrathoracic pressure is in these implementations defined as the integral of the PPG2 relative blood volume waveform with respect to time (the time period may be one breath, or other time periods or durations/intervals may be used). In other words, the indication of intrathoracic pressure represents, or is defined as, the relative change in blood volume during a predefined number (can be one or more) respiratory cycles/breaths.

S1010 comprises determining a severity of a respiratory condition based on the determined indication of intrathoracic pressure. This determination can be performed in a variety of ways.

In one example, with reference to FIGS. 8 and 9, the indication of intrathoracic pressure can be used to classify or characterize an obstructive respiratory condition of an unseen subject or patient based on comparing the indication of intrathoracic pressure to one or more thresholds. For example, a patient can be classified with a mild obstruction or a severe obstruction based on the indication of intrathoracic pressure. In some specific examples, a mapping or relationship between the indication of intrathoracic pressure and spirometry measurements can be provided, such that the individual can be classified as having an FEV1/FVC ratio of>0.45 or <0.45. In other examples, any other suitable thresholds can be used. This approach is a discrete classification, which can be performed by way of a look-up table, or predefined range or mapping the to one or more thresholds.

S1010 can therefore comprise comparing the determined indication of intrathoracic pressure to one or more predefined indications (of intrathoracic pressure), each having an associated severity. These predefined indications can act as/represent the one or more thresholds. Step 1010 can also comprise classifying the determined indication of intrathoracic pressure with the severity based on the comparing. For example, an indication of intrathoracic pressure below a predefined indication can be classified with a first severity, and an indication of intrathoracic pressure above the predefined indication can be classified with a second severity.

This classification approach applies to all obstructive breathing disorders, such as COPD, asthma, and others such as cystic fibrosis, though it will be understood that the range of FEV1/FVC ratios and/or associated thresholds may be different for different diseases. Depending on the thresholds used or selected, determining a severity of a respiratory condition can comprise determining whether an obstructive respiratory condition is present at all; e.g. an indication of intrathoracic pressure below/above (as appropriate) a predefined threshold can indicate no obstruction. When an obstruction is detected, operation S1010 can further comprise classifying a severity of the obstructive respiratory condition by classifying the determined indication of intrathoracic pressure with the severity based on comparing the determined indication of intrathoracic pressure to one or more predefined indications (i.e. to one or more thresholds) having an associated severity.

In another example, with reference to FIG. 7, a continuous mapping between the indication of intrathoracic pressure and severity may be used to monitor changes in severity in real time or almost real time. The mapping may be a formula or transform, derived from theory or experiment. In some examples, the mapping is an empirically derived curve/mapping. For example, a formula could be fit to the four left most data points of FIG. 7 to provide an individualised relationship between indication of intrathoracic pressure and severity. S1010 may therefore comprise calculating the severity based on one or more predetermined relationships between an indication of intrathoracic pressure and a severity. Such a predetermined relationship may be determined by experimentation and/or theory (such as modelling). Machine learning may also be used to determine the one or more predetermined relationships between an indication of intrathoracic pressure and a severity. This approach can allow for periodic or continuous monitoring of a respiratory condition, allowing gradual changes in the condition with time to be observed. More fine-grained monitoring may therefore be provided than with a discrete approach to mapping the indication and the severity.

By repeating method 1000 on new data and periodically (or continually) updating the indication of intrathoracic pressure, the severity of a condition can be monitored over time. An output can be provided, or caused to be provided, to a user based on the determined severity. For example, when a condition worsens, a user may be notified at the apparatus. The output can be graphical or audible or haptic. The output can be a message or alert to the user, or any other type of notification. Alternatively or additionally, the output can be provided to another computing device. The other computing device may be associated with a user of the apparatus, or may be associated with one or more third parties, such as a doctor or carer. The output can be provided at the apparatus performing method 1000, or the apparatus 200 can cause the output to be provided, e.g. by providing data to another computing device, such as a mobile phone, to cause the output.

The following numbered examples are disclosed:
Example 1. An apparatus for determining a severity of a respiratory condition, the apparatus comprising: means for receiving data indicative of an intensity of light detected during a photoplethysmography measurement; means for determining an indication of intrathoracic pressure based on the received data; and means for determining a severity of a respiratory condition based on the determined indication of intrathoracic pressure.
Example 2. The apparatus of example 1, wherein the means for determining an indication of intrathoracic pressure based on the received data comprises: means for determining a signal indicative of a relative change in blood volume from the received data; and means for integrating the signal indicative of a relative change in blood volume with respect to time to determine the indication of intrathoracic pressure.
Example 3. The apparatus of example 2, wherein the means for determining a signal indicative of a relative change in blood volume comprises means for signal conditioning, including: means for normalising the received data; means for zeroing the normalised data; means for inverting the zeroed data to determine the signal indicative of a relative change in blood volume.
Example 4. The apparatus of any preceding example, wherein the means for determining a severity of a respiratory condition based on the determined indication of intrathoracic pressure comprises: means for comparing the determined indication of intrathoracic pressure to one or more predefined indications having an associated severity; means for classifying the determined indication of intrathoracic pressure with the severity based on the comparing.
Example 5. The apparatus of any of examples 1 to 3, wherein the means for determining a severity of a respiratory condition based on the determined indication of intrathoracic pressure comprises: means for calculating the severity, the calculation based on one or more predetermined relationships between indication of intrathoracic pressure and severity.
Example 6. The apparatus of any preceding example, further comprising means for causing, based on the determined severity, an output to be provided.
Example 7. The apparatus of example 6, further comprising means for providing the output to a user of the apparatus.
Example 8. The apparatus of any preceding example, wherein the respiratory condition is an obstructive respiratory condition.
Example 9. The apparatus of example 8, wherein the obstructive respiratory condition is one of: COPD, asthma, or cystic fibrosis.
Example 10. An apparatus for detecting an obstructive respiratory condition, the apparatus comprising: means for receiving data indicative of an intensity of light detected during a photoplethysmography measurement over a respiratory cycle; means for determining a first indication of intrathoracic pressure during an expiration phase of the respiratory cycle based on the received data; means for determining a second indication of intrathoracic pressure during an inspiration phase of the respiratory cycle based on the received data; and means for detecting an obstructive respiratory condition based on a ratio of the first and second indications of intrathoracic pressure.
Example 11. A system comprising: the apparatus of any preceding example; and a sensor for photoplethysmography measurement, the sensor comprising: a light source configured to emit light; a photodetector configured to detect reflected light, wherein the data indicative of an intensity of light detected during a photoplethysmography measurement is indicative of the detected reflected light.
Example 12. The system of example 11, wherein the sensor is configured to be worn in an ear of a user.
Example 13. The system of example 11 or example 12, further comprising one or more additional sensors configured to output one or more sensing signals, wherein the means for determining an indication of intrathoracic pressure is further configured to determine the indication of intrathoracic pressure based on the one or more sensing signals.
Example 14. The system of example 13, wherein the one or more sensing signals are indicative of at least one of: ECG, blood pressure, heart rate variability (HRV), motion, temperature, or audio.
Example 15. A method performed by one or more processors of an apparatus, the method comprising: receiving data indicative of an intensity of light detected during a photoplethysmography measurement; determining an indication of intrathoracic pressure based on the received data; determining a severity of a respiratory condition based on the determined indication of intrathoracic pressure.
Example 16. The method of example 15, further comprising repeatedly receiving, at periodic intervals, subsequent data indicative of an intensity of light detected during a subsequent photoplethysmography measurement, the method further comprising, for each subsequent data: determining an updated indication of intrathoracic pressure based on the subsequent data; and updating the severity of the respiratory condition based on the updated indication of intrathoracic pressure.
Example 17. The method of example 15 or example 16, further comprising causing, based on the severity, an output to be provided.
Example 18. The method of example 17, wherein providing an output comprising

outputting an alert to a user.

Example 19. A method for detecting an obstructive respiratory condition, the method comprising: receiving data indicative of an intensity of light detected during a photoplethysmography measurement over a respiratory cycle; determining a first indication of intrathoracic pressure during an expiration phase of the respiratory cycle based on the received data; determining a second indication of intrathoracic pressure during an inspiration phase of the respiratory cycle based on the received data; detecting an obstructive respiratory condition based on a ratio of the first and second indications of intrathoracic pressure.

Example 20. A computer program comprising instructions for causing an apparatus to perform the method of any of examples 15 to 19.

1. An apparatus for determining a severity of a respiratory condition, the apparatus comprising:
means for receiving data indicative of an intensity of light detected during a photoplethysmography measurement;
means for determining an indication of intrathoracic pressure based on the received data; and
means for determining a severity of a respiratory condition based on the determined indication of intrathoracic pressure.

2. The apparatus of claim 1, wherein the means for determining an indication of intrathoracic pressure based on the received data comprises:
means for determining a signal indicative of a relative change in blood volume from the received data; and
means for integrating the signal indicative of a relative change in blood volume with respect to time to determine the indication of intrathoracic pressure.

3. The apparatus of claim 2, wherein the means for determining a signal indicative of a relative change in blood volume comprises means for signal conditioning, including:
means for normalising the received data;
means for zeroing the normalised data;
means for inverting the zeroed data to determine the signal indicative of a relative change in blood volume.

4. The apparatus of claim 1, wherein the means for determining a severity of a respiratory condition based on the determined indication of intrathoracic pressure comprises:
means for comparing the determined indication of intrathoracic pressure to one or more predefined indications having an associated severity;
means for classifying the determined indication of intrathoracic pressure with the severity based on the comparing.

5. The apparatus of claim 1, wherein the means for determining a severity of a respiratory condition based on the determined indication of intrathoracic pressure comprises:
means for calculating the severity, the calculation based on one or more predetermined relationships between indication of intrathoracic pressure and severity.

6. The apparatus of claim 1, further comprising means for causing, based on the determined severity, an output to be provided.

7. The apparatus of claim 6, further comprising means for providing the output to a user of the apparatus.

8. The apparatus of claim 1, wherein the respiratory condition is an obstructive respiratory condition, optionally, wherein the obstructive respiratory condition is one of: COPD, asthma, or cystic fibrosis.

9. The apparatus of claim 5, wherein the obstructive respiratory condition is COPD, and wherein the severity is classified based on a predetermined relationship between the relative change in blood volume and a volume during forced expiration in one second, FEV1.

10. The apparatus of claim 9, wherein the severity is classified based on a predetermined relationship between the relative change in blood volume and a ratio of the volume during forced expiration in one second, FEV1, against a forced vital capacity, FEC.

11. An apparatus for detecting an obstructive respiratory condition, the apparatus comprising:
means for receiving data indicative of an intensity of light detected during a photoplethysmography measurement over a respiratory cycle;
means for determining a first indication of intrathoracic pressure during an expiration phase of the respiratory cycle based on the received data;
means for determining a second indication of intrathoracic pressure during an inspiration phase of the respiratory cycle based on the received data; and
means for detecting an obstructive respiratory condition based on a ratio of the first and second indications of intrathoracic pressure.

12. (canceled)

13. (canceled)

14. (canceled)

15. (canceled)

16. A method performed by one or more processors of an apparatus, the method comprising:
receiving data indicative of an intensity of light detected during a photoplethysmography measurement;
determining an indication of intrathoracic pressure based on the received data;
determining a severity of a respiratory condition based on the determined indication of intrathoracic pressure.

17. The method of claim 16, further comprising repeatedly receiving, at periodic intervals, subsequent data indicative of an intensity of light detected during a subsequent photoplethysmography measurement, the method further comprising, for each subsequent data:
determining an updated indication of intrathoracic pressure based on the subsequent data; and
updating the severity of the respiratory condition based on the updated indication of intrathoracic pressure.

18. The method of claim 16, further comprising causing, based on the severity, an output to be provided, optionally, wherein providing an output comprising outputting an alert to a user.

19. (canceled)

20. (canceled)
